(19) United States
(12) Patent Application Publication
(10) Pub. No.: US 2022/0241306 A1
(43) Pub. Date: Aug. 4, 2022

(54) CHITIN ANALOGS FOR THE TREATMENT OF KIDNEY DISEASES

(71) Applicant: AyuVis Research, Inc., Fort Worth, TX (US)

(72) Inventors: Suchismita Acharya, Grapevine, TX (US); Santosh K. Panda, Maryland Heights, MO (US)

(21) Appl. No.: 17/591,280

(22) Filed: Feb. 2, 2022

Related U.S. Application Data
(60) Provisional application No. 63/145,666, filed on Feb. 4, 2021.

(51) Int. Cl. A61K 31/7034

(52) U.S. Cl. CPC A61K 31/7034

(57) ABSTRACT

The present invention includes compositions and methods for preventing or treating a patient for kidney injury comprising administering an effective amount of a compound of Formula I:

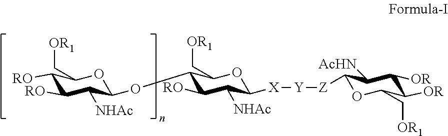

where n=0-5; X=NH, O, S, or CH₂; Y=phenyl, or a phenyl group substituted with at least one methyl, a phenyl group substituted with at least one nitro, a phenyl group substituted with at least one nitrogen, a phenyl group substituted with at least one boron, or aryl, substituted aryl, heteroaryl, four to six membered cycloalkyl, four to six membered heterocycloalkyl; R=H, C(O)R₂, SO₂R₂; R₁=H, C(O)R₂, SO₂R₂; R₂=ethyl, methyl, isopropyl, n-propyl, t-butyl, n-butyl, NH₂, NR₃R₄, R₃, R₄=ethyl, methyl, isopropyl, n-propyl, t-butyl, n-butyl, three to six membered cycloalkyl; and Z=NH, O, S, CH₂, or none.

### CROSS-REFERENCE TO RELATED APPLICATIONS

This application claims priority to U.S. Provisional Application Ser. No. 63/145,666, filed Feb. 4, 2021, the entire contents of which are incorporated herein by reference.

### TECHNICAL FIELD OF THE INVENTION

The present invention relates in general to the field of preventing and treating acute kidney injury, and more particularly, to compositions and methods to prevent or treat acute kidney injury.

### STATEMENT OF FEDERALLY FUNDED RESEARCH

None.

### BACKGROUND OF THE INVENTION

Without limiting the scope of the invention, its background is described in connection with acute kidney injury.

Acute kidney injury (AKI) is a critical clinical problem with a high mortality rate and can occur after an acute injury or as a silent event, being identified only after its occurrence by the onset of progressive azotemia. Rhabdomyolysis-induced AKI, also termed “crush” kidney injury, develops after skeletal muscle trauma related to physical, thermal, ischemic, infective, metabolic, or toxic causes, releasing toxic doses of myoglobin and other intracellular proteins into the circulation. Approximately 10 to 50% of patients suffering from rhabdomyolysis develop some degree of AKI, and although interventions have improved, the mortality rate may still be as high as 8%. Although the pathogenesis of glycerol-induced AKI is complex and incompletely understood, apoptosis, inflammation, and oxidative stress (Rosenberger et al., 2008; Kim et al., 2010; Wei et al., 2011) have been implicated. Recent studies have demonstrated that timely prophylactic and/or early therapeutic interventions (either pre- or coadministration) ameliorated glycerol-induced AKI.

A need remains for novel compositions and method of preventing and/or treating acute kidney injury.

### SUMMARY OF THE INVENTION

In one embodiment, the present invention includes a method of preventing or treating a patient for acute kidney injury comprising administering an effective amount of a compound of Formula I:

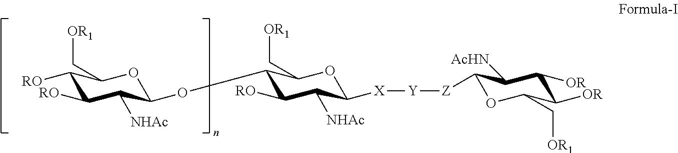

where n=0-5; X=NH, O, S, or CH₂; Y=phenyl, or a phenyl group substituted with at least one methyl, a phenyl group substituted with at least one nitro, a phenyl group substituted with at least one nitrogen, a phenyl group substituted with at least one boron, or aryl, substituted aryl, heteroaryl, four to six membered cycloalkyl, four to six membered heterocycloalkyl; R=H, C(O)R₂, SO₂R₂; R₁=H, C(O)R₂, SO₂R2; R₂=ethyl, methyl, isopropyl, n-propyl, t-butyl, n-butyl, NH₂, NR₃R₄, R₃, R₄=ethyl, methyl, isopropyl, n-propyl, t-butyl, n-butyl, three to six membered cycloalkyl; and Z=NH, O, S, CH₂, or none. In one aspect, the compound is administered after developing acute kidney injury. In another aspect, the patient is a human patient that is elderly, pregnant, a surgical patient, or has been exposed to a nephrotoxic agent, wherein the nephrotoxic agent is a drug or chemical capable of causing acute kidney injury. In another aspect, the acute kidney injury is characterized by a serum creatinine level of at least 1.5 times baseline, wherein baseline refers to the patient's serum creatinine level no more than 7 days prior; the serum creatinine level is 1.5 to 1.9 times baseline, and the acute kidney injury is characterized by an increase in serum creatinine of at least 0.3 mg/dL; or the serum creatinine level is 3.0 or more times baseline. In another aspect, the acute kidney injury is characterized by an increase in serum creatinine of at least 0.3 mg/dL; the acute kidney injury is characterized by an increase in serum creatinine of at least 0.4 mg/dL. In another aspect, the acute kidney injury is characterized by a glomerular filtration rate of 60-89 mL/min/1.73 m²; or the acute kidney injury is characterized by a glomerular filtration rate of less than 90 mL/min/1.73 m². In another aspect, the glomerular filtration rate is 60-89 mL/min/1.73 m², wherein the acute kidney injury is characterized by the patient having a urine output of less than 0.5 mL/Kg over 6 hours; or the glomerular filtration rate is less than 15 mL/min/1.73 m². In another aspect, the acute kidney injury is characterized by the patient having a urine output of less than 0.5 mL/Kg over 6 hours; the urine output is less than 0.3 mL/Kg over 12 hours, or wherein the acute kidney injury is characterized by the patient having anuria for 12 or more hours.

In another aspect, the compound is:

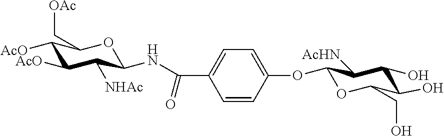

(Compound 2, AVR-123), or a pharmaceutically acceptable salt thereof. In another aspect, the compound is:

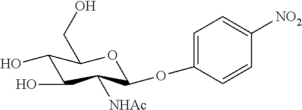

(Compound 8, AVR-48), or a pharmaceutically acceptable salt thereof. In another aspect, the patient is suffering from acute interstitial nephritis, acute glomerular renal disease, acute vasculitic renal disease, ischemia, toxic injury, prerenal axotemia, azotemia, or acute postrenal destructive nephropathy. In another aspect, the patient has diabetes, underlying renal insufficiency, nephritic syndrome, atherosclerotic disease, sepsis, hypotension, hypoxia, myoglobinuria-hematuri a, or liver disease. In another aspect, the compound is formulated into a pharmaceutical composition adapted for intravenous, oral, enteral, parenteral, intrarenal, or subcutaneous administration. In another aspect, the compound is not formulated for intramuscular administration or injected intramuscularly. In another aspect, the method further comprises providing the compound prior to surgery or a treatment that causes acute kidney injury.

In another embodiment, the present invention includes a method of preventing or treating a patient for acute kidney injury comprising administering an effective amount of a TLR4/TLR2 modulatory chitin analog to the human patient comprising: identifying the patient in need of prevention or treatment of the acute kidney injury; and providing a therapeutically effective amount of the TLR4/TLR2 modulatory chitin analog of Formula I:

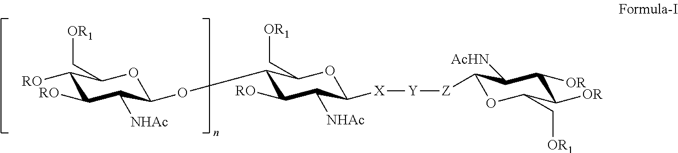

where n=0-5; X=NH, O, S, CH₂; Y=phenyl, or a phenyl group substituted with at least one methyl, a phenyl group substituted with at least one nitro, a phenyl group substituted with at least one nitrogen, a phenyl group substituted with at least one boron, or aryl, substituted aryl, heteroaryl, four to six membered cycloalkyl, four to six membered heterocycloalkyl; R=H, C(O)R₂, SO₂R₂; R₁=H, C(O)R₂, SO₂R₂; R₂=ethyl, methyl, isopropyl, n-propyl, t-butyl, n-butyl, NH₂, NR₃R₄, R₃, R₄=ethyl, methyl, isopropyl, n-propyl, t-butyl, n-butyl, three to six membered cycloalkyl; and Z=NH, O, S, CH₂, or none.

In one aspect, the TLR4/TLR2 modulatory chitin analog is administered after developing acute kidney injury. In another aspect, the patient is a human patient that is elderly, pregnant, a surgical patient, or has been exposed to a nephrotoxic agent, wherein the nephrotoxic agent is a drug or chemical capable of causing acute kidney injury. In another aspect, the acute kidney injury is characterized by a serum creatinine level of at least 1.5 times baseline, wherein baseline refers to the patient's serum creatinine level no more than 7 days prior; the serum creatinine level is 1.5 to 1.9 times baseline, and the acute kidney injury is characterized by an increase in serum creatinine of at least 0.3 mg/dL; or the serum creatinine level is 3.0 or more times baseline. In another aspect, the acute kidney injury is characterized by an increase in serum creatinine of at least 0.3 mg/dL; the acute kidney injury is characterized by an increase in serum creatinine of at least 0.4 mg/dL. In another aspect, the acute kidney injury is characterized by a glomerular filtration rate of 60-89 mL/min/1.73 m²; or the acute kidney injury is characterized by a glomerular filtration rate of less than 90 mL/min/1.73 m². In another aspect, the glomerular filtration rate is 60-89 mL/min/1.73 m², wherein the acute kidney injury is characterized by the patient having a urine output of less than 0.5 mL/Kg over 6 hours; or the glomerular filtration rate is less than 15 mL/min/1.73 m². In another aspect, the acute kidney injury is characterized by the patient having a urine output of less than 0.5 mL/Kg over 6 hours; the urine output is less than 0.3 mL/Kg over 12 hours, or wherein the acute kidney injury is characterized by the patient having anuria for 12 or more hours. In another aspect, the TLR4/TLR2 mediated macrophage modulatory chitin analog is:

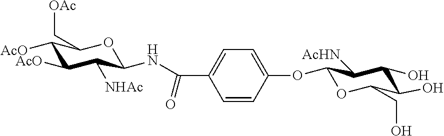

(Compound 2, AVR-123), or a pharmaceutically acceptable salt thereof.

In another aspect, the TLR4/TLR2 mediated macrophage modulatory chitin analog is:

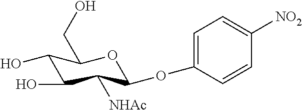

(Compound 8, AVR-48), or a pharmaceutically acceptable salt thereof. In another aspect, the patient is suffering from acute interstitial nephritis, acute glomerular renal disease, acute vasculitic renal disease, ischemia, toxic injury, prerenal axotemia, azotemia, or acute postrenal destructive nephropathy. In another aspect, the patient has diabetes, underlying renal insufficiency, nephritic syndrome, atherosclerotic disease, sepsis, hypotension, hypoxia, myoglobinuria-hematuria, or liver disease. In another aspect, the TLR4/TLR2 modulatory chitin analog is formulated into a pharmaceutical composition adapted for intravenous, oral, enteral, parenteral, intrarenal, or subcutaneous administration. In another aspect, the TLR4/TLR2 modulatory chitin analog is not formulated for intramuscular administration or injected intramuscularly. In another aspect, the method further comprises providing the compound prior to surgery or a treatment that causes acute kidney injury.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the change in serum creatinine level after treatment with AVR-48 and AVR-123 as determined by creatine assay kit (Cayman chemical) following manufacture instructions. N=4-7 mice. *p=0.02, **p=0.00′7, and ***p=0.0004, One-way ANOVA, Dunnett's multiple comparison test.

FIG. 2 is a graph that shows the change in blood urea nitrogen (BUN) level after AVR-48 treatment as determined by using the urea assay kit (Cayman chemical) following manufacture instructions. N=4-7 mice, each sample run in duplicates. ****p<0.0001, One-way ANOVA, Dunnett's multiple comparison test.

FIG. 3 is a graph that shows the total proteinuria level as determined from urine by using albustix proteinuria strip (Amazon) following manufacture instructions. N=3-4 mice, each sample run in duplicates. **p=0.0012, ****p<0.0001, One-way ANOVA, Dunnett's multiple comparison test.

FIG. 4 is a graph that shows the level of the inflammatory cytokine TNF-α in whole kidney tissue with or without AVR-48 treatment as determined by RT-PCR experiment. TNF-α was found to be significantly elevated in glycerol-treated mice where treatment with AVR-48 significantly decreased the level. N=3-5 mice, each sample run in duplicates. *p=0.01**p=0.0036, One-way ANOVA, Dunnett's multiple comparison test.

FIG. 5 is a graph that shows the level of the inflammatory cytokine IL-1β in whole kidney tissue with or without AVR-48 treatment as determined by RT-PCR experiment. IL-1β was found to be significantly elevated in glycerol-treated mice where treatment with AVR-48 significantly decreased the level. N=3-5 mice, each sample run in duplicates. **p=0.009***p=0.0004, One-way ANOVA, Dunnett's multiple comparison test.

FIGS. 6A and 6B are graphs that show the level of the anti-inflammatory cytokine IL-10 in the whole kidney tissue (FIG. 6A) and in kidney cortex tissue (FIG. 6B) with or without AVR-48 treatment as determined by RT-PCR experiment. IL-10 was found to be significantly high whole kidney tissue in AVR-48 treated group. In kidney cortex tissue. An increase in IL-10 in glycerol treated group is a natural response to inflammation and AVR-48 treatment decreased the level and brought it back close to normal. N=3-4 mice, each sample run in duplicates. **p<0.01, ***p<0.001, One-way ANOVA, Dunnett's multiple comparison test.

FIGS. 7A and 7B are graphs that show the level of antioxidant enzyme hemeoxygenase-1 (OH-1) and NGAL in kidney cortical tissue. An increase in OH-1 in kidney tissue shows a response to inflammation and AVR-48 decreased the level significantly (FIG. 7A). NGAL is increased and considered as a marker of kidney injury which was decreased after treatment with AVR-48 (FIG. 7B). N=3-4 mice, each sample run in duplicates. **p<0.01, **p<0.01, ***p<0.001, One-way ANOVA, Dunnett's multiple comparison test.

FIG. 8 is a graph showing an increase in cyclooxygenase-2 (Cox-2) enzyme after glycerol injection and AVR-48 treatment decreased significantly Cox-2 mRNA level in the whole kidney tissue.

FIG. 9 is a graph showing the survival curve of the mouse in a glycerol-induced rhabdomyolysis model when dosed after 3 h of glycerol administration. There was found to be a significant difference in survival (p<0.05) between glycerol+saline and glycerol+AVR-48 (10 mg/kg/dose) mouse groups.

FIG. 10 shows representative H/E staining of mouse whole kidney tissue. Treatment with AVR-48 showed significant protection of kidney tissues against glycerol-induced damage. N=5.

FIGS. 11A and 11B compare renal cortical histology from the preterm lamb treated with 3.0 mg/Kg of AVR-48 (FIG. 11A) versus the vehicle-treated lamb (saline; FIG. 11B). Renal cortical histology is normal in the 3.0 mg/Kg AVR48-treated lamb. Numerous glomeruli and open proximal and distal tubules (FIG. 11A). In comparison, the vehicle-treated kidney has glomeruli that appeared clumped and tubules are mostly closed, suggestive of acute renal injury.

FIG. 12 shows decreased BUN at day 10 followed by 7 days of AVR-48 treatment while in invasive mechanical ventilator. Fetal lamb is term lamb control. N=2-3

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Kidney diseases are typically classified as either chronic or acute. Acute kidney injury (AKI) is commonly associated with bacterial infection, sepsis or ischemia-reperfusion injury (I/R that can transition to chronic renal disease), chronic kidney disease (CKD) that typically results from diabetic complications, hypertension, obesity, and autoimmunity. The initiating events that promote renal disease can be quite different; however, AKI can lead to CKD, and, if unchecked, both can lead to end-stage renal disease (ESRD). Importantly, inflammation and immune system activation represent a common underlying characteristic for both AKI and CKD (Imig and Ryan 2013).

Inflammation and immune system activation are important causal factors in the development of both acute and chronic renal disease. Several key components of innate immunity have been implicated in the progression of renal disease including the complement system, toll-like receptors (TLRs), dendritic cells, macrophages, natural killer (NK) cells, and inflammatory cytokines. When cytokines are involved in the pathogenesis of renal disease, they contribute by upregulating endothelial cell adhesion molecules and chemokines that further promote renal immune cell infiltration. In addition, signaling pathways activated by many cytokines increase Nuclear factor kappa-light-chain enhancer of activated B cells (NF-κB) activation, a transcription factor that further promotes a pro-inflammatory phenotype (Sanz, Justo et al. 2008, Sanz, Sanchez-Nino et al. 2010). Indeed, NF-κB expression and/or activation are increased in the kidneys from patients with glomerulonephritis (Zheng, Sinniah et al. 2006) diabetic nephropathy (Mezzano, Aros et al. 2004) and AKI (Loverre, Ditonno et al. 2004). One of the consequences of this inflammatory cycle is to drive the development of local oxidative stress that enhances renal injury and impairs both renal tubular and renal hemodynamic function; Toll-like receptors (TLRs) are a group of cell surface proteins that serve as pattern-recognition receptors that typically bind to microbial pathogens and initiate an inflammatory response. TLRs have been implicated in both AKI and CKD. For example, TLR4 knockout mice were protected against cisplatin-induced AKI, and nonspecific TLR inhibition with chloroquine protected against renal dysfunction in a mouse model of sepsis (Yasuda, Leelahavanichkul et al. 2008, Zhang, Ramesh et al. 2008). In addition to AKI, TLR2 and TLR4 directly correlate with renal disease severity and inflammatory markers suggesting a pathogenic role for TLRs in CKD in humans. Renal TLR4 expression was significantly associated with the pro-inflammatory marker MCP-1 and the profibrotic molecule TGF-β1 in kidney biopsies from patients with CKD, suggesting that increased expression of TLR4 is an important feature of CKD (Lepenies, Eardley et al. 2011). Similarly, TLR-2 is associated with the inflammatory response of non-dialysed and dialysed CKD patients (Koc, Toprak et al. 2011).

Macrophages, phagocytic cells derived from monocytes, are located in peripheral tissues and act as important mediators of inflammation and immune modulation. They contribute both to normal physiological function and pathophysiology and are prevalent in the kidneys of humans with chronic renal disease (Ferrario, Castiglione et al. 1985). Macrophages can be activated by immune complexes associated with complement or by cells of the adaptive immune system (T lymphocytes and their cytokines). In the setting of renal disease, macrophage activation often occurs secondarily to complement activation or effector T cells activated by antigens not specific to the kidney suggesting that macrophages may not be prominent initiators of renal disease (Duffield 2010). Nevertheless, both AKI and chronic renal diseases including autoimmune-mediated glomerulonephritis are associated with increased macrophage numbers in the kidney (Sean Eardley and Cockwell 2005).

It is worth noting that there are a variety of different macrophage subpopulations. Although it is an oversimplification, they are most commonly classified as either M1 or M2. The subpopulation associated with renal disease is the classically activated proinflammatory M1 macrophages that are activated by inflammatory cytokines such as interferon gamma (IFN-γ) or tumor necrosis factor alpha (TNF-α) (Mosser and Edwards 2008) and are derived from cells of the innate or adaptive immune systems. Classically activated macrophages release inflammatory cytokines, promote oxidative stress, and the development of renal fibrosis (Ricardo, van Goor et al. 2008). The causal role for classically activated macrophages in nephritis is supported by studies showing that macrophage depletion or inhibition of monocyte chemoattractant protein (MCP-1) reduces renal macrophages and protects the kidney. While macrophages have an important role to scavenge cellular debris, increased renal infiltration of macrophages can tip the balance toward causing local injury and inflammation. The injury and inflammation are mediated by the release of macrophage-derived inflammatory cytokines like interleukin (IL)-1β, IL-6, IL-23, and the generation of reactive oxygen/nitrogen species, each of which have been implicated in impaired renal function (Kielar, John et al. 2005, Ghee, Han et al. 2008).

The immunomodulatory actions of T-regulatory (Tregs) are proposed to occur by releasing cytokines like transforming growth factor beta (TGF-β) and IL-10 that can inhibit the release of M1 cytokines. Down regulation of Treg cells and/or dysfunction of Treg cells promote autoimmune disease and inflammation. The protective role for Tregs against renal injury is supported by evidence showing that expanding the Treg population can delay the onset of renal injury and inflammation associated with autoimmune-induced nephritis (Tucci, Stucci et al. 2010).

Jung et al. demonstrated that IL-10 may exert protective effects through the induction of lipocalin-2, also known as NGAL, in a rat model of renal ischemia (Jung, Sola et al. 2012). Overexpressing IL-10 via adoptive transfer reduced inflammation and kidney injury dependent on increased expression of NGAL, its receptor. Zhang et al (Zhang, Garg et al. 2015) have found higher IL-10 to be associated with decreased risk of all-cause mortality following cardiac surgery after adjustment for clinical and demographic factors, as well as renal function. Several studies have reported the influence of IL-10 in attenuating inflammation and kidney injury in animal models of acute GN, (Kitching, Katerelos et al. 2002, Choi, Kim et al. 2003) CKD, cisplatin nephrotoxicity and ischemic injury (Deng, Kohda et al. 2001). Further, animal studies in a variety of other disease settings have also demonstrated similar protective properties of IL-10 and have even reported that the cytokine creates an environment conducive for regenerative adult wound healing.

Glucosamine is the monomer of the biopolymer chitosan and N-acetyl glucosamine (NAG) is monomer of the biopolymer chitin. The effects of chitosan on renal function in patients with chronic renal failure have been studied (Jing, Li et al. 1997). The effects of chitosan were investigated on eighty patients with renal failure undergoing long-term stable hemodialysis treatment. The patients were tested after a control treatment period of 1 week. Half were fed 30 chitosan tablets (45 mg chitosan/tablet) three times a day. Ingestion of chitosan effectively reduced total serum cholesterol levels (from 10.14+/−4.40 to 5.82+/−2.19 mM) and increased serum hemoglobin levels (from 58.2+/−12.1 to 68+/−9.0 g L-1). Significant reductions in urea and creatinine levels in serum were observed after 4 weeks of chitosan ingestion. The feeling of physical strength, the appetite and the sleep of patients in the treatment group had improved significantly after 12 weeks of ingestion, compared with those of patients in the control group. During the treatment period, no clinically problematic symptoms were observed. These data suggest that chitosan might be effective treatment for renal failure patients, however at a higher dose of 135 mg/kg per day.

Glucosamine performs its physiological effects through the N-acetyl glucosamine (NAG and in this form, it is embedded in glycosaminoglycans and glycoproteins of biological membranes, including the glomerular basement membrane (Morita et al., 2008 Dalirfardouei et al., 2016). Therefore, potentially NAG has a more expressed nephroprotective effect because of the direct mechanism of action.

The present invention includes composition and methods that use chitin and NAG derived macrophage modulators via interacting with toll like receptors (TLRs), e.g., TLR4 and TLR2 (TLR4/TLR2), to simultaneously increase IL-10, suppress kidney inflammation while decrease kidney toxicity useful in AKI, CKI, acute interstitial nephritis, acute glomerular renal disease, acute vasculitic renal disease, ischemia, toxic injury, prerenal axotemia, azotemia, or acute postrenal destructive nephropathy elderly, pregnant, a surgical patient, or has been exposed to a nephrotoxic agent, and/or wherein the nephrotoxic agent is a drug or chemical capable of causing acute kidney injury. One such compound has the following Formula (Formula I):

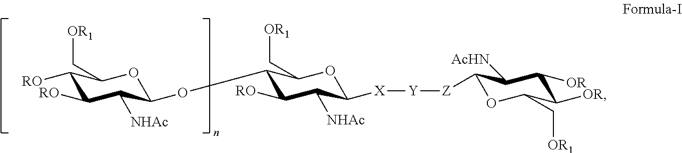

where n=0-5;
X=NH, O, S, or CH₂;
Y=four to six membered cycloalkyl, a phenyl group substituted with at least one methyl, a phenyl group substituted with at least one nitro, a phenyl group substituted with at least one nitrogen, a phenyl group substituted with at least one boron, aryl, substituted aryl, heteroaryl, cycloalkyl;
R=H, C(O)R₂, SO₂R₂;
R₁=H, C(O)R₂, SO₂R₂; and
R₂=Alkyl, substituted alkyl, aryl, substituted aryl, NHR₃
R₃=H, ethyl, methyl, isopropyl, n-propyl, t-butyl, n-butyl,
Z=NH, O, S, CH₂ or none.

In some embodiments, the compounds of the present disclosure are incorporated into parenteral formulations. The term parenteral as used herein includes subcutaneous, intravenous, intramuscular, and intra-arterial injections with a variety of infusion techniques. Intra-arterial and intravenous injection as used herein includes administration through catheters. Preferred for certain indications are methods of administration that allow rapid access to the tissue or organ being treated, such as intravenous injections for the treatment of endotoxemia or sepsis.

The compounds of the present disclosure will be administered in dosages which will provide suitable inhibition or activation of TLR of the target cells; generally, these dosages are, preferably between 0.25-50 mg/patient, or from 1.0-100 mg/patient or from 5.0-200 mg/patient or from 100-500 mg/patient, more preferably, between 0.25-50 mg/patient and most preferably, between 1.0-100 mg/patient. The dosages are preferably once a day for 28 days, more preferably twice a day for 14 days or most preferably 3 times a day for 7days.

Pharmaceutical compositions containing the active ingredient may be in any form suitable for the intended method of administration. Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

Aqueous suspensions of the compounds of the present invention contain the active materials in admixture with excipients suitable for the manufacture of aqueous suspensions. Such excipients include a suspending agent, such as sodium carboxymethylcellulose, methylcellulose, hydroxypropylmethyl cellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadeaethyleneoxycetanol), a condensation product of ethylene oxide with a partial ester derived from a fatty acid and a hexitol anhydride (e.g., polyoxyethylene sorbitan mono-oleate). The aqueous suspension may also contain one or more preservative such as ethyl of n-propyl p-hydroxybenzoate.

The pharmaceutical compositions of the invention can be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenteral-acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the formulation comprises PLA or PLGA microparticles and may be further mixed with Na₂HPO₄, hydroxypropyl methylcellulose, polysorbate 80, sodium chloride, and/or edetate disodium.

Formulations suitable for parenteral administration include aqueous and non-aqueous isotonic sterile injection solutions which may contain anti-oxidants, buffers, bacteriostats and solutes which render the formation isotonic with the blood of the intended recipient; and aqueous and non-aqueous sterile suspensions which may include suspending agents and thickening agents. The formulations may be presented in unit-dose or multi-dose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders of the kind previously described.

It will be understood, however, that the specific dose level for any particular patient will depend on a variety of factors including the activity of the specific compound employed; the age, body weight, general health, and sex of the individual being treated; the time and route of administration; the rate of excretion; other drugs which have previously been administered; and the severity of the particular disease undergoing therapy.

In some embodiments the compositions of the present disclosure also contain from about 80% to about 99.5%, preferably from about 90% or 95% to about 98.5% of a compatible non-aqueous pharmaceutically acceptable topical vehicle. Some vehicles are described in U.S. Pat. 4,621,075, which is incorporated herein for this disclosure. Although it is preferred that these vehicles be free of water, the compositions of the present invention may contain up to about 5% water without significant adverse effects on the formation of the desired gels. These non-aqueous vehicle components are also well-known in the pharmaceutical arts, and they include (but are not limited to) short chain alcohols and ketones and emollients, such as hydrocarbon oils and waxes, lanolin and lanolin derivatives, silicone oils, monoglyceride, diglyceride, and triglyceride esters, fatty alcohols, alkyl and alkenyl esters of fatty acids, alkyl and alkenyl diesters of dicarboxylic acids, polyhydric alcohols and their ether and ester derivatives; wax esters and beeswax derivatives. Preferred vehicles incorporate methanol, ethanol, n-propanol, isopropanol, butanol, polypropylene glycol, polyethylene glycol and mixtures of these components. Particularly preferred vehicles include ethanol, n-propanol and butanol, especially ethanol. These preferred solvents may also be combined with other components, such as diisopropyl sebacate, isopropyl myristate, methyl laurate, silicone, glycerine and mixtures of these components, to provide non-aqueous vehicles which are also useful in the present invention. Of these additional components, diisopropyl sebacate is especially useful. In fact, preferred vehicles include mixtures of ethanol and diisopropyl sebacate in ratios, by weight, of from about 4:1 to about 1:4. Preferred vehicles contain from about 15% to about 35% diisopropyl sebacate and from about 65% to about 85% ethanol.

Compositions of the present invention may additionally contain, at their art-established usage levels, compatible adjunct components conventionally used in the formulation of topical pharmaceutical compositions. These adjunct components may include, but are not limited to, pharmaceutically-active materials (such as supplementary antimicrobial or anti-inflammatory ingredients, e.g., steroids) or ingredients used to enhance the formulation itself (such as excipients, dyes, perfumes, skin penetration enhancers, stabilizers, preservatives, and antioxidants). Examples of such agents include the pharmaceutically-acceptable acidic carboxy polymers, such as the Carbopol compounds commercially available from B. F. Goodrich Chemicals, Cleveland, Ohio.

In one embodiment, the compounds of the present invention may be formulate into a cream, lotion or gel packaged in a common trigger spray container will be firmly adhered to the area of interest as a regular cream does after it is sprayed out from the container. This is described in WO 98/51273, which is incorporated herein by reference. Accordingly, in one aspect, the present disclosure provides a pharmaceutical that can be incorporated into a non-aerosol spray composition for topical application, which comprises the compounds as described herein alone or in combination. The compounds are present in an amount in the range of 0.1% to 20% or in some embodiments from 1 to 15% by weight, or in some embodiments from 2 to 10% by weight of cream, lotion or gel. The compounds of the present invention can be incorporated into a neutral hydrophilic matrix cream, lotion or gel. In a first preferred embodiment, the cream or lotion matrix for topical application is characterized by polyoxyethylene alkyl ethers. In a second preferred embodiment, the gel is characterized by high molecular weight polymer of cross-linked acrylic acid. Polyoxyethylene alkyl ethers are non-ionic surfactants widely used in pharmaceutical topical formulations and cosmetics primarily as emulsifying agents for water-in-oil and oil-in-water emulsions. It is characterized in this invention as a base for non-aerosol trigger sprayable cream or lotion. Cross-linked acrylic acid polymer (Carbomer) employed to form the gel is another object of this invention.

Mouse model for acute kidney injury. Eight to ten weeks old male C57 BL/6J mice (n=5-7 per group, male and female) were utilized to induce acute kidney injury. Animals were randomized before treatment. Littermates or co-housed animals were chosen for this study. The mice were kept without water but unlimited access to chow for −24 hrs. In first study, nine ml per kg body weight of 1:1 diluted glycerol was intramuscularly injected followed by intraperitoneal administration of either compound 2 (5 mg/kg) or compound 8 (2.5 mg/kg body weight) at 1, 6 and 24 hrs post glycerol dosing. In a second study, AVR-48 was injected either at 5.0 mg/kg/dose or 10 mg/kg/dose IP after 3 h of glycerol administration and was dosed 2/day for 3 days (total 7 doses) and survival was monitored till 8 days.

Proteinuria was measured in urine at 24 hrs before sacrifice of the animals by an albustix proteinuria strip.

The mice were sacrificed, and bloods were collected by heart puncture. Serum samples were stored at −80° C. for future use. Kidney, spleens, lungs and liver were immediately fixed in 10% buffered formalin for histopathological analysis. Part of the kidney were snap frozen for RNA isolation.

RNA isolation and RT-PCR. RNA was isolated from snap-frozen kidney samples by using RNA plus micro kit (Qiagen) according to manufacturer's instruction. c-DNA were prepared by Quanta-Bio-C-DNA kit. RT-PCR was performed using following primers for various genes. The expression of various genes was analyzed in comparison to GAPDH.

AVR compounds ameliorates clinical symptoms of acute kidney injury.

To evaluate the effect of AVR-48 and AVR-123 on acute kidney injury, the inventors measured the vital parameters of kidney injury, i.e., levels of creatinine and blood urea nitrogen

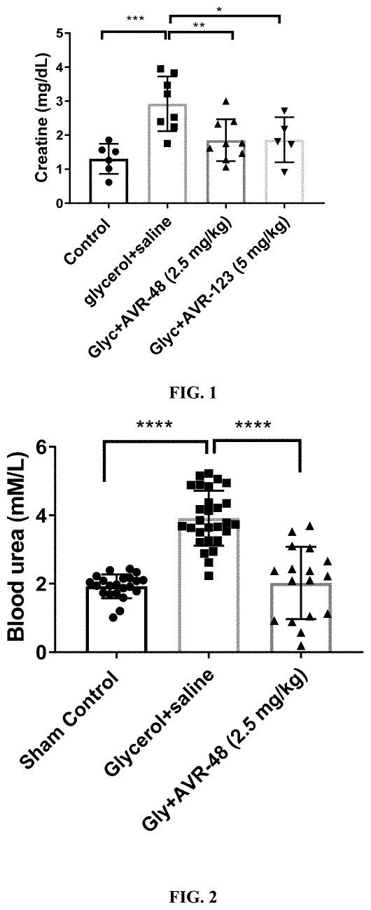

(BUN) in the serum of animals treated with AVR-48 and AVR-123 along with glycerol treated positive controls. PBS treated animals were considered as sham. Glycerol induced creatinine and BUN levels were significantly reduced in the serum upon the compound's treatment (FIGS. 1 and 2). FIG. 1 shows the change in serum creatinine level as determined by using the creatine assay kit (Cayman chemical) following manufacture instructions. N=4-7 mice. *p=0.02, **p=0.00′7, and ***p=0.0004, One-way ANOVA, Dunnett's multiple comparison test. FIG. 2 shows the change in blood urea nitrogen (BUN) level after AVR-48 using the urea assay kit (Cayman chemical) following manufacture instructions. N=4-7 mice, each sample run in duplicates. ****p<0.0001, One-way ANOVA, Dunnett's multiple comparison test.

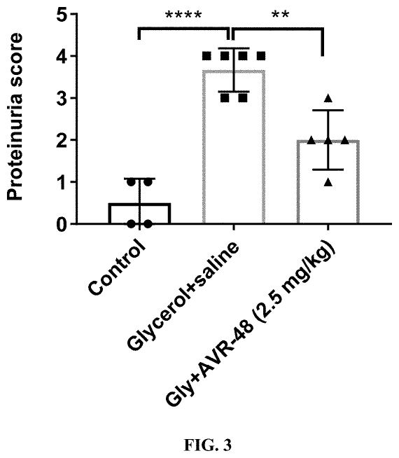

Glycerol treated mice have high proteinuria levels as compared to sham control group. In contrast, decreased proteinuria levels were observed upon AVR-48 treatment (FIG. 3). FIG. 3 shows the total proteinuria level as determined from urine by using albustix proteinuria strip (Amazon) following manufacture instructions. N=3-4 mice, each sample run in duplicates. **p=0.0012, ****p<0.0001, One-way ANOVA, Dunnett's multiple comparison test.

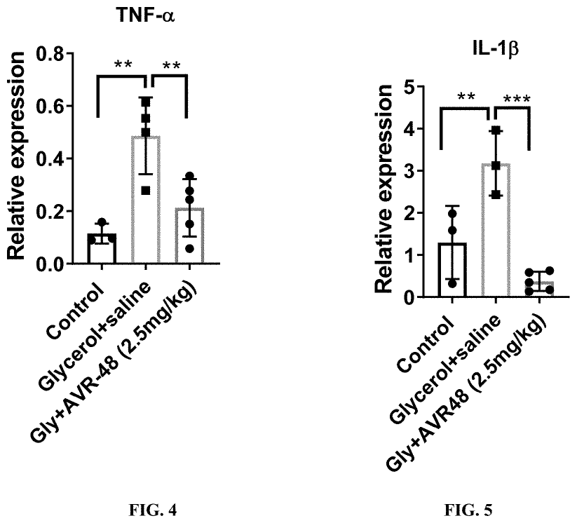

AVR-48 reduced glycerol-induced/treated inflammation in the kidney. Higher expression of pro-inflammatory cytokines is a signature of acute kidney injury. To access the role of AVR-48 on the glycerol-induced pro-inflammatory cytokine production, the inventors performed RT-PCR analysis of kidney tissues. Significant elevation of inflammatory cytokines TNF-α, IL-1β was observed in the glycerol-treated mice. Inhibition of both TNF-α(FIG. 4) and IL-1β (FIG. 5) were observed after AVR-48 treatment. FIG. 4 shows the level of the inflammatory cytokine TNF-α in kidney tissue with or without AVR-48 treatment as determined by RT-PCR experiment. TNF-α was found to be significantly elevated in glycerol-treated mice where treatment with AVR-48 significantly decreased the level. N=3-5 mice, each sample run in duplicates. *p=0.01**p=0.0036, One-way ANOVA, Dunnett's multiple comparison test. FIG. 5 shows the level of the inflammatory cytokine IL-1β in kidney tissue with or without AVR-48 treatment as determined by RT-PCR experiment. IL-1β was found to be significantly elevated in glycerol treated mice where treatment with AVR-48 significantly decreased the level. N=3-5 mice, each sample run in duplicates. **p=0.009***p=0.0004, One-way ANOVA, Dunnett's multiple comparison test.

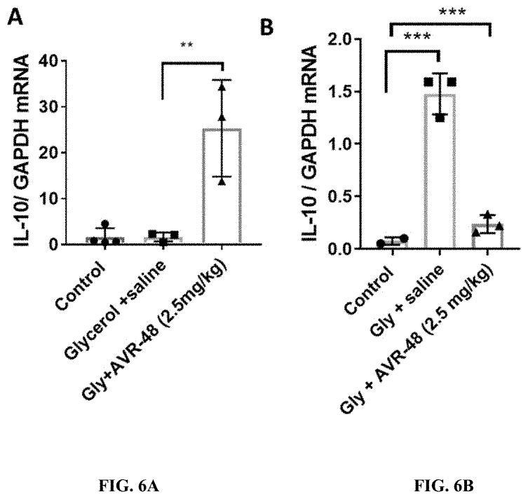

Interestingly, IL-10 levels were up regulated upon AVR-48 treatment (FIGS. 6A and 6B) confirming the inventors' previous observations that AVR compounds have capability to quieten the excess inflammation and up regulate the anti-inflammatory cytokine IL-10 (Das, Panda et al. 2019). FIGS. 6A and 6B are graphs that show the level of the anti-inflammatory cytokine IL-10 in the whole kidney tissue (FIG. 6A) and in kidney cortex tissue (FIG. 6B) with or without AVR-48 treatment as determined by RT-PCR experiment. IL-10 was found to be significantly high whole kidney tissue in AVR-48 treated group. In kidney cortex tissue. An increase in IL-10 in glycerol treated group seems a natural response to inflammation and AVR-48 treatment decreased the level and brought it back close to normal. N=3-4 mice, each sample run in duplicates. **p<0.01, ***p<0.001, One-way ANOVA, Dunnett's multiple comparison test.

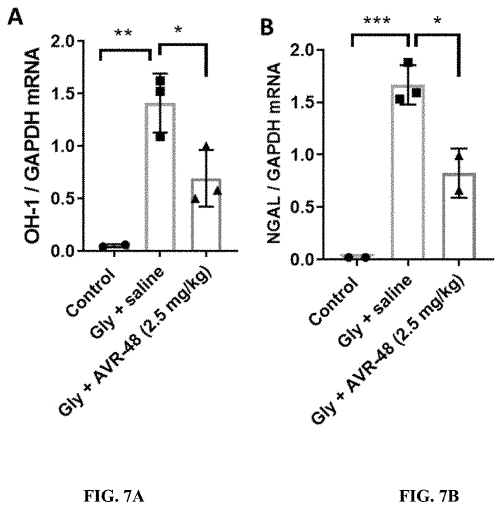

FIG. 7A and 7B are graphs that show the level of antioxidant enzyme hemeoxygenase-1 (OH-1) and NGAL in kidney cortical tissue. An increase in OH-1 in kidney tissue shows the response to inflammation and AVR-48 decreased the level significantly (FIG. 7A). NGAL is increased and considered as a marker of kidney injury which was decreased after treatment with AVR-48 (7B). N=3-4 mice, each sample run in duplicates. *p<0.05, **p<0.01, ***p<0.001, One-way ANOVA, Dunnett's multiple comparison test.

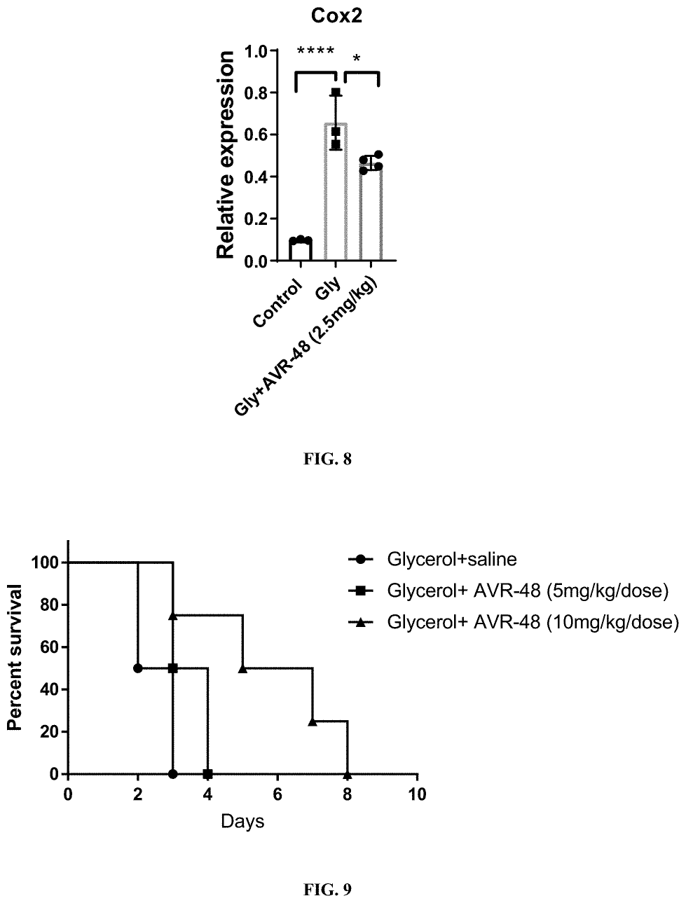

FIG. 8 is a graph showing increase in cyclooxygenase-2 (Cox-2) enzyme after glycerol injection and AVR-48 treatment decreased significantly Cox-2 mRNA level in the whole kidney tissue. *p<0.05, ****p<0.0001, One-way ANOVA, Dunnett's multiple comparison test.

FIG. 9 is a graph showing survival curve of the mouse in a glycerol induced rhabdomyolysis model when dosed after 3 h of glycerol administration. There found to be significant difference in survival (p<0.05) between glycerol+saline and glycerol+AVR-48 (10 mg/kg/dose) group of mouse.

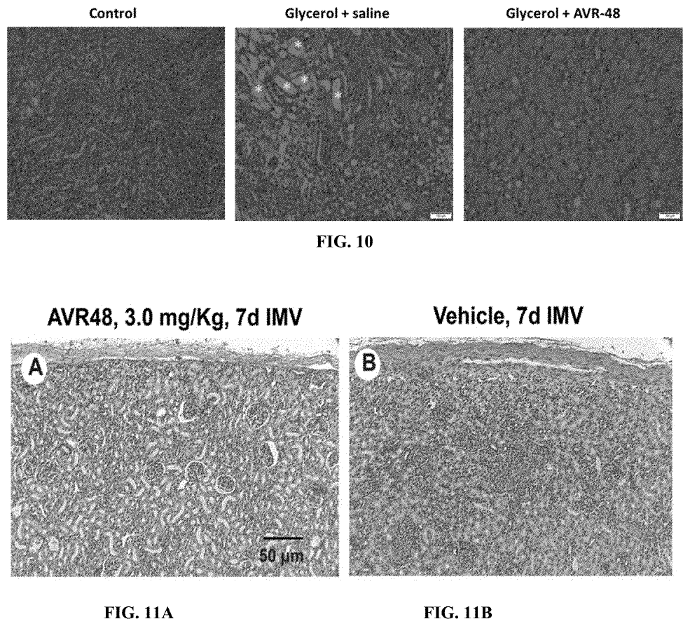

FIG. 10 shows representative H/E staining of mouse whole kidney tissue. Treatment with AVR-48 showed significant protection of kidney tissues against glycerol induced damage. N=5.

Preterm lamb model of mechanical ventilator induced kidney injury: Next, the inventors tested the drug exposure to kidney, lung and other organs in a higher animal model that mimics preterm (PT) human infants and demonstrate the efficacy of the compounds taught herein to prevent AKI via IV dosing. A unique PT lamb model, developed by Dr. Albertine (University of Utah), was used to emulate the clinical setting for PT human infants with respiratory failure related to premature birth before the lungs are mature enough to support extra-uterine life.

A dose range-finding study was conducted with the goal of treating 2-3 PT lambs with either a vehicle control or AVR-48 (compound 8, 3mg/kg/dose) by twice daily intravenous infusions during 6 to 7 days of mechanical ventilation, followed by transition to noninvasive respiratory support (NIS) for 3 days, for a total of 10 days of management of these PT lambs. AVR-48 was not given during the period of noninvasive respiratory support to assess short-term persistence of effect of AVR-48. Detailed methods have been published previously by Reyburn et al. and Null et al (Reyburn, Li et al. 2008, Null, Alvord et al. 2014), relevant portions incorporated herein by reference. All protocols have been adhered to APS/NIH guidelines for animal research and have been approved by the Institution Animal Care and Use Committees (IACUC) at the University of Utah.

FIGS. 11A and 11B compare renal cortical histology from the preterm lamb treated with 3.0 mg/Kg of AVR-48 (FIG. 11A) versus the vehicle-treated lamb (saline; FIG. 11B). Renal cortical histology is normal in the 3.0 mg/Kg AVR-48-treated lamb. Numerous glomeruli and open proximal and distal tubules (FIG. 11A). In comparison, the vehicle-treated kidney has glomeruli that appeared clumped and tubules are mostly closed, suggestive of acute renal injury.

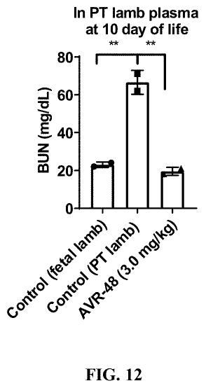

FIG. 12 shows decreased BUN at day 10 followed by 7 days of AVR-48 treatment while in invasive mechanical ventilator. Fetal lamb is term lamb control. N=2-3, **p<0.01, *** student t test (2-tailed), Dunnett's multiple comparison test.

In certain embodiments, the present invention includes compositions and methods for preventing or treating a patient for acute kidney injury comprising, consisting essentially of, or consisting of: administering an effective amount of a compound of Formula I:

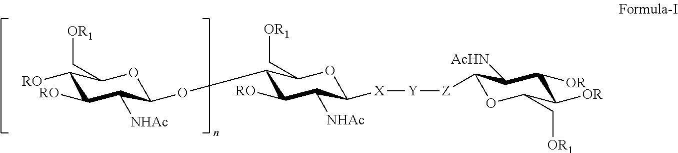

where n=0-5; X=NH, O, S, or CH₂; Y=phenyl, or a phenyl group substituted with at least one methyl, a phenyl group substituted with at least one nitro, a phenyl group substituted with at least one nitrogen, a phenyl group substituted with at least one boron, or aryl, substituted aryl, heteroaryl, four to six membered cycloalkyl, four to six membered heterocycloalkyl; R=H, C(O)R₂, SO₂R₂; R₁=H, C(O)R₂, SO₂R₂; R₂=ethyl, methyl, isopropyl, n-propyl, t-butyl, n-butyl, NH₂, NR₃R₄, R₃, R₄=ethyl, methyl, isopropyl, n-propyl, t-butyl, n-butyl, three to six membered cycloalkyl; and Z=NH, O, S, CH₂, or none.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure.

Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Choi, Y. K., Y. J. Kim, H. S. Park, K. Choi, S. G. Paik, Y. I. Lee and J. G. Park (2003). “Suppression of glomerulosclerosis by adenovirus-mediated IL-10 expression in the kidney.” Gene Ther 10(7): 559-568.
Das, P., S. K. Panda, B. Agarwal, S. Behera, S. M. Ali, M. E. Pulse, J. S. Solomkin, S. M. Opal, V. Bhandari and S. Acharya (2019). “Novel Chitohexaose Analog Protects Young and Aged mice from CLP Induced Polymicrobial Sepsis.” Scientific Reports 9(1): 2904.
Deng, J., Y. Kohda, H. Chiao, Y. Wang, X. Hu, S. M. Hewitt, T. Miyaji, P. McLeroy, B.

Nibhanupudy, S. Li and R. A. Star (2001). “Interleukin-10 inhibits ischemic and cisplatin-induced acute renal injury.” Kidney Int 60(6): 2118-2128.
Duffield, J. S. (2010). “Macrophages and immunologic inflammation of the kidney.” Semin Nephrol 30(3): 234-254.
Ferrario, F., A. Castiglione, G. Colasanti, G. Barbiano di Belgioioso, S. Bertoli and G. D'Amico (1985). “The detection of monocytes in human glomerulonephritis.” Kidney Int 28(3): 513-519.
Ghee, J. Y., D. H. Han, H. K. Song, W. Y. Kim, S. H. Kim, H. E. Yoon, B. S. Choi, Y. S. Kim, J. Kim and C. W. Yang (2008). “The role of macrophage in the pathogenesis of chronic cyclosporine-induced nephropathy.” Nephrol Dial Transplant 23(12): 4061-4069.
Imig, J. D. and M. J. Ryan (2013). “Immune and inflammatory role in renal disease.” Comprehensive Physiology 3(2): 957-976.
Jing, S. B., L. Li, D. Ji, Y. Takiguchi and T. Yamaguchi (1997). “Effect of chitosan on renal function in patients with chronic renal failure.” J Pharm Pharmacol 49(7): 721-723.
Jung, M., A. Sola, J. Hughes, D. C. Kluth, E. Vinuesa, J. L. Villas, A. Perez-Ladaga and G. Hotter (2012). “Infusion of IL-10-expressing cells protects against renal ischemia through induction of lipocalin-2.” Kidney Int 81(10): 969-982.
Kielar, M. L., R. John, M. Bennett, J. A. Richardson, J. M. Shelton, L. Chen, D. R. Jeyarajah, X. J. Zhou, H. Zhou, B. Chiquett, G. T. Nagami and C. Y. Lu (2005). “Maladaptive role of IL-6 in ischemic acute renal failure.” J Am Soc Nephrol 16(11): 3315-3325.
Kitching, A. R., M. Katerelos, S. J. Mudge, P. G. Tipping, D. A. Power and S. R. Holdsworth (2002). “Interleukin-10 inhibits experimental mesangial proliferative glomerulonephritis.” Clin Exp Immunol 128(1): 36-43.
Koc, M., A. Toprak, H. Arikan, Z. Odabasi, Y. Elbir, A. Tulunay, E. Asicioglu, E. Eksioglu-Demiralp, G. Glorieux, R. Vanholder and E. Akoglu (2011). “Toll-like receptor expression in monocytes in patients with chronic kidney disease and haemodialysis: relation with inflammation.” Nephrol Dial Transplant 26(3): 955-963.
Lepenies, J., K. S. Eardley, T. Kienitz, M. Hewison, T. Ihl, P. M. Stewart, P. Cockwell and M. Quinkler (2011). “Renal TLR4 mRNA expression correlates with inflammatory marker MCP-1 and profibrotic molecule TGF-β₁ in patients with chronic kidney disease.” Nephron Clin Pract 119(2): c97-c104.
Loverre, A., P. Ditonno, A. Crovace, L. Gesualdo, E. Ranieri, P. Pontrelli, G. Stallone, B. Infante, A. Schena, S. Di Paolo, C. Capobianco, M. Ursi, S. Palazzo, M. Battaglia, F. P. Selvaggi, F. P. Schena and G. Grandaliano (2004). “Ischemia-reperfusion induces glomerular and tubular activation of proinflammatory and antiapoptotic pathways: differential modulation by rapamycin.” J Am Soc Nephrol 15(10): 2675-2686.
Mezzano, S., C. Aros, A. Droguett, M. E. Burgos, L. Ardiles, C. Flores, H. Schneider, M. Ruiz-Ortega and J. Egido (2004). “NF-kappaB activation and overexpression of regulated genes in human diabetic nephropathy.” Nephrol Dial Transplant 19(10): 2505-2512.
Mosser, D. M. and J. P. Edwards (2008). “Exploring the full spectrum of macrophage activation.” Nat Rev Immunol 8(12): 958-969.
Null, D. M., J. Alvord, W. Leavitt, A. Wint, M. J. Dahl, A. P. Presson, R. H. Lane, R. J. DiGeronimo, B. A. Yoder and K. H. Albertine (2014). “High-frequency nasal ventilation for 21 d maintains gas exchange with lower respiratory pressures and promotes alveolarization in preterm lambs.” Pediatr Res 75(4): 507-516.
Reyburn, B., M. Li, D. B. Metcalfe, N. J. Kroll, J. Alvord, A. Wint, M. J. Dahl, J. Sun, L. Dong, Z. M. Wang, C. Callaway, R. A. McKnight, L. Moyer-Mileur, B. A. Yoder, D. M. Null, R. H. Lane and K. H. Albertine (2008). “Nasal ventilation alters mesenchymal cell turnover and improves alveolarization in preterm lambs.” Am J Respir Crit Care Med 178(4): 407-418.
Ricardo, S. D., H. van Goor and A. A. Eddy (2008). “Macrophage diversity in renal injury and repair.” J Clin Invest 118(11): 3522-3530.
Sanz, A. B., P. Justo, M. D. Sanchez-Niño, L. M. Blanco-Colio, J. A. Winkles, M. Kreztler, A. Jakubowski, J. Blanco, J. Egido, M. Ruiz-Ortega and A. Ortiz (2008). “The cytokine TWEAK modulates renal tubulointerstitial inflammation.” J Am Soc Nephrol 19(4): 695-703.
Sanz, A. B., M. D. Sanchez-Niño, A. M. Ramos, J. A. Moreno, B. Santamaria, M. Ruiz-Ortega, J. Egido and A. Ortiz (2010). “NF-kappaB in renal inflammation.” J Am Soc Nephrol 21(8): 1254-1262.
Sean Eardley, K. and P. Cockwell (2005). “Macrophages and progressive tubulointerstitial disease.” Kidney Int 68(2): 437-455.
Tucci, M., S. Stucci, S. Strippoli and F. Silvestris (2010). “Cytokine overproduction, T-cell activation, and defective T-regulatory functions promote nephritis in systemic lupus erythematosus.” J Biomed Biotechnol 2010: 457146.
Yasuda, H., A. Leelahavanichkul, S. Tsunoda, J. W. Dear, Y. Takahashi, S. Ito, X. Hu, H. Zhou, K. Doi, R. Childs, D. M. Klinman, P. S. Yuen and R. A. Star (2008). “Chloroquine and inhibition of Toll-like receptor 9 protect from sepsis-induced acute kidney injury.” Am J Physiol Renal Physiol 294(5): F1050-1058.
Zhang, B., G. Ramesh, S. Uematsu, S. Akira and W. B. Reeves (2008). “TLR4 signaling mediates inflammation and tissue injury in nephrotoxicity.” J Am Soc Nephrol 19(5): 923-932.
Zhang, W. R., A. X. Garg, S. G. Coca, P. J. Devereaux, J. Eikelboom, P. Kaysak, E. McArthur, H. Thiessen-Philbrook, C. Shortt, M. Shlipak, R. Whitlock and C. R. Parikh (2015). “Plasma IL-6 and IL-10 Concentrations Predict AKI and Long-Term Mortality in Adults after Cardiac Surgery.” Journal of the American Society of Nephrology 26(12): 3123-3132.
Zheng, L., R. Sinniah and S. I. Hsu (2006). “In situ glomerular expression of activated NF-kappaB in human lupus nephritis and other non-proliferative proteinuric glomerulopathy.” Virchows Arch 448(2): 172-183.

What is claimed is:

1. A method of preventing or treating a patient for acute kidney injury comprising administering an effective amount of a compound of Formula I: 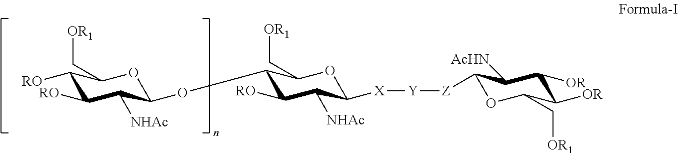
where n=0-5;
X=NH, O, S, or CH₂;
Y=phenyl, or a phenyl group substituted with at least one methyl, a phenyl group substituted with at least one nitro, a phenyl group substituted with at least one nitrogen, a phenyl group substituted with at least one boron, or aryl, substituted aryl, heteroaryl, four to six membered cycloalkyl, four to six membered heterocycloalkyl;
R=H, C(O)R₂, SO₂R₂;
R₁=H, C(O)R₂, SO₂R₂;
R₂=ethyl, methyl, isopropyl, n-propyl, t-butyl, n-butyl, NH₂, NR₃R₄;
R₃, R₄=ethyl, methyl, isopropyl, n-propyl, t-butyl, n-butyl, three to six membered cycloalkyl; and
Z=NH, O, S, CH₂, or none.

2. The method of claim 1, wherein the compound is administered at least one of: after developing acute kidney injury; or prior to surgery or a treatment that causes acute kidney injury; or has been exposed to a nephrotoxic agent, wherein the nephrotoxic agent is a drug or chemical capable of causing acute kidney injury.

3. The method of claim 1, wherein the acute kidney injury is characterized by a serum creatinine level of at least 1.5 times baseline, wherein baseline refers to the patient's serum creatinine level no more than 7 days prior; the serum creatinine level is 1.5 to 1.9 times baseline, and the acute kidney injury is characterized by an increase in serum creatinine of at least 0.3 mg/dL; or the serum creatinine level is 3.0 or more times baseline; the acute kidney injury is characterized by at least one of: an increase in serum creatinine of at least 0.3 mg/dL; the acute kidney injury is characterized by an increase in serum creatinine of at least 0.4 mg/dL; the acute kidney injury is characterized by a glomerular filtration rate of 60-89 mL/min/1.73 m²; or the acute kidney injury is characterized by a glomerular filtration rate of less than 90 mL/min/1.73 m² or the acute kidney injury is characterized by the patient having a urine output of less than 0.5 mL/Kg over 6 hours; the urine output is less than 0.3 mL/Kg over 12 hours, or the acute kidney injury is characterized by the patient having anuria for 12 or more hours.

4. The method of claim 3, wherein the glomerular filtration rate is 60-89 mL/min/1.73 m², wherein the acute kidney injury is characterized by the patient having a urine output of less than 0.5 mL/Kg over 6 hours; or the glomerular filtration rate is less than 15 mL/min/1.73 m².

5. The method of claim 1, wherein the compound is: 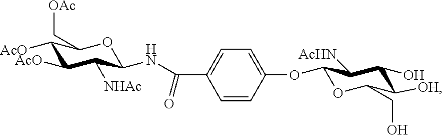
or a pharmaceutically acceptable salt thereof.

6. The method of claim 1, wherein the compound is: 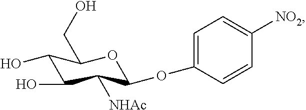
or a pharmaceutically acceptable salt thereof.

7. The method of claim 1, wherein the patient is suffering from acute interstitial nephritis, acute glomerular renal disease, acute vasculitic renal disease, ischemia, toxic injury, prerenal axotemia, azotemia, or acute postrenal destructive nephropathy.

8. The method of claim 1, wherein the patient has diabetes, underlying renal insufficiency, nephritic syndrome, atherosclerotic disease, sepsis, hypotension, hypoxia, myoglobinuria-hematuria, or liver disease.

9. The method of claim 1, wherein the compound is formulated into a pharmaceutical composition adapted for intravenous, oral, enteral, parenteral, intrarenal, or subcutaneous administration.

10. The method of claim 1, wherein the compound is not formulated for intramuscular administration or injected intramuscularly.

11. A method of preventing or treating a patient for acute kidney injury comprising administering an effective amount of a TLR4/TLR2 modulatory chitin analog to the human patient comprising:
identifying the patient in need of prevention or treatment of the acute kidney injury; and
providing a therapeutically effective amount of the TLR4/TLR2 modulatory chitin analog of Formula I: 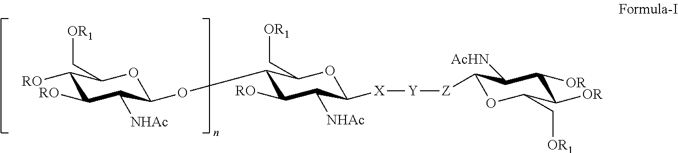
where n=0-5;
X=NH, O, S, CH₂;
Y=phenyl, or a phenyl group substituted with at least one methyl, a phenyl group substituted with at least one nitro, a phenyl group substituted with at least one nitrogen, a phenyl group substituted with at least one boron, or aryl, substituted aryl, heteroaryl, four to six membered cycloalkyl, four to six membered heterocycloalkyl;
R=H, C(O)R₂, SO₂R₂;
R₁=H, C(O)R₂, SO₂R₂;
R₂=ethyl, methyl, isopropyl, n-propyl, t-butyl, n-butyl, NH₂, NR₃R₄;
R₃, R₄=ethyl, methyl, isopropyl, n-propyl, t-butyl, n-butyl, three to six membered cycloalkyl; and
Z=NH, O, S, CH₂, or none.

12. The method of claim 11, wherein the TLR4/TLR2 modulatory chitin analog is administered at least one of: after developing acute kidney injury; or prior to surgery or a treatment that causes acute kidney injury; or has been exposed to a nephrotoxic agent, wherein the nephrotoxic agent is a drug or chemical capable of causing acute kidney injury.

13. The method of claim 11, wherein the acute kidney injury is at least one of: characterized by a serum creatinine level of at least 1.5 times baseline, wherein baseline refers to the patient's serum creatinine level no more than 7 days prior; the serum creatinine level is 1.5 to 1.9 times baseline, and the acute kidney injury is characterized by an increase in serum creatinine of at least 0.3 mg/dL; or the serum creatinine level is 3.0 or more times baseline; the acute kidney injury is characterized by an increase in serum creatinine of at least 0.3 mg/dL; the acute kidney injury is characterized by an increase in serum creatinine of at least 0.4 mg/dL; the acute kidney injury is characterized by a glomerular filtration rate of 60-89 mL/min/1.73 m²; or the acute kidney injury is characterized by a glomerular filtration rate of less than 90 mL/min/1.73 m²; or the acute kidney injury is characterized by the patient having a urine output of less than 0.5 mL/Kg over 6 hours; the urine output is less than 0.3 mL/Kg over 12 hours, or wherein the acute kidney injury is characterized by the patient having anuria for 12 or more hours.

14. The method of claim 13, wherein the glomerular filtration rate is 60-89 mL/min/1.73 m², wherein the acute kidney injury is characterized by the patient having a urine output of less than 0.5 mL/Kg over 6 hours; or the glomerular filtration rate is less than 15 mL/min/1.73 m².

15. The method of claim 11, wherein the TLR4/TLR2 modulatory chitin analog is: 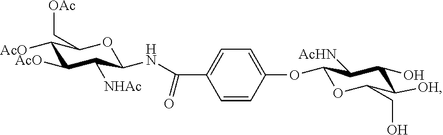
or a pharmaceutically acceptable salt thereof.

16. The method of claim 11, wherein the TLR4/TLR2 modulatory chitin analog is: 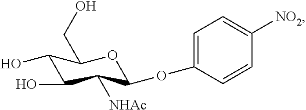
or a pharmaceutically acceptable salt thereof.

17. The method of claim 11, wherein the patient is suffering from acute interstitial nephritis, acute glomerular renal disease, acute vasculitic renal disease, ischemia, toxic injury, prerenal axotemia, azotemia, or acute postrenal destructive nephropathy.

18. The method of claim 11, wherein the patient has diabetes, underlying renal insufficiency, nephritic syndrome, atherosclerotic disease, sepsis, hypotension, hypoxia, myoglobinuria-hematuria, or liver disease.

19. The method of claim 11, wherein the TLR4/TLR2 modulatory chitin analog is formulated into a pharmaceutical composition adapted for intravenous, oral, enteral, parenteral, intrarenal, or subcutaneous administration.

20. The method of claim 11, wherein the TLR4/TLR2 modulatory chitin analog is not formulated for intramuscular administration or injected intramuscularly.
